(19) United States
(12) Patent Application Publication
(10) Pub. No.: US 2014/0257098 A1
(43) Pub. Date: Sep. 11, 2014

(54) SYSTEMS AND METHODS FOR DETECTION OF CANCER IN WOMEN

(71) Applicant: Giuseppe Del Priore, Indianapolis, IN (US)

(72) Inventor: Giuseppe Del Priore, Indianapolis, IN (US)

(21) Appl. No.: 14/197,837

(22) Filed: Mar. 5, 2014

Related U.S. Application Data
(60) Provisional application No. 61/772,976, filed on Mar. 5, 2013.
(60) Provisional application No. 61/821,986, filed on May 10, 2013.

(51) Int. Cl. A61B 5/00; A61K 49/00; A61M 5/00; A61M 25/10; A61B 8/08; A61B 10/02

(52) U.S. Cl. CPC A61B 5/4325; A61B 8/085; A61B 10/0291; A61M 5/007; A61M 25/10; A61K 49/006; A61M 2025/105; A61M 2025/1052
USPC 600/434; 600/443; 600/563

(57) ABSTRACT

Devices and methods for screening for ovarian and endometrial cancer are disclosed. The device includes a catheter positioned to enter an endometrial cavity of the patient. The device can include a transvaginal probe configured to be inserted into a vagina of the patient, and a needle both coupled to the transvaginal probe. The needle can be is positioned to enter a cul-de-sac of the patient when the transvaginal probe is inserted into the vagina, and into the cul-de-sac, and the needle is configured to collect the fluid from the cul-de-sac. The catheter can also configured to deliver fluid into the endometrial cavity. The fluid can also be a dye injected into the endometrial cavity and absorbed by the endometrial carcinoma and that drains into at least one sentinel lymph node. The sentinel lymph node can be identified to allow for dissection and further examination for micrometastasis.

### CROSS-REFERENCE TO RELATED APPLICATIONS

This application claims the benefit of U.S. Provisional Patent Application Ser. Nos. 61/772,976 filed on Mar. 5, 2013 and 61/821,986 filed on May 10, 2013, each of which is incorporated herein by reference in its entirety.

### BACKGROUND OF THE INVENTION

The present application is directed to systems, devices, and methods for use in the early detection of ovarian cancer, for the collection of cells from the endometrial cavity and the fallopian tubes that can be analyzed for screening ovarian cancer, and for sentinel lymph node detection in endometrial carcinoma.

Gynecological cancers, including ovarian cancer and endometrial carcinoma pose a major health risk to women. Endometrial carcinoma, for example, is the most common malignancy of the female genital tract in developed countries. In the United States, between 1987-2006, the number of women newly diagnosed as having endometrial cancer increased from 35,000 to 41,200 and the number of deaths rose from 2900 to 7350, a 153% increase. Additionally, an estimated 21,880 women were diagnosed with ovarian cancer, and 13,850 died from the disease in 2010. The overall five-year survival rate from ovarian cancer decreases by stage of diagnosis, from 92.1% at the earliest stage to 11.6% at stage IV disease. Most patients present with advanced disease and, as a result, have a poor prognosis. Early diagnosis is therefore important.

Despite the importance of early diagnosis, currently, there are no screening tests or known early detection strategies for ovarian cancer. The only known way to definitively confirm or rule out ovarian cancer is through surgery. A recent study found that, out of 182 patients referred to gynecologic oncology for suspected ovarian cancer, more than 30% of the patients had to undergo the risks of surgery only to confirm no significant findings of cancer. Of those women who are actually diagnosed, more than 70% are at an advanced stage at the time of diagnosis, partly because the women must visit a multitude of physicians and specialists prior to referral to gynecologic oncology for suspected ovarian cancer and subsequent surgery. The five-year survival rate of women with advanced stage disease is less than 40%, whereas for women with early stage disease at diagnosis, the five-year survival rate is greater than 80%.

Similarly, available methods for testing and screening patients with endometrial cancer are limited. Patients with endometrial cancer often undergo surgical staging, which includes peritoneal cytology, total hysterectomy with bilateral salpingooophorectomy, and pelvic and paraaortic lymphadenectomy. However, these recommendations and the surgical treatment of patients with a preoperative diagnosis of low-risk stage I endometrioid endometrial cancer remain an area of significant debate, with a wide variation in the intraoperative management of the retroperitoneal lymph nodes. This is partly due to the fact that the incidence of extrauterine disease in women with clinical stage I endometrial cancer has been demonstrated to be approximately 20%, and the surgery needed to remove many lymph nodes to find the one that may contain cancer incurs risks for the patient. As a result, complete pelvic and paraaortic lymphadenectomy may incur increased morbidity without any benefit for approximately 80% of women with clinical stage I endometrial cancer.

Currently, preoperative and intraoperative prediction of who would and would not benefit from a lymphadenectomy is still inaccurate and unreliable. Current imaging modalities are restricted in their ability to detect micrometastatic lymph node spread. For example, MRI and CT imaging modalities are not sensitive for diagnosing lymph node metastases by size criteria. Many studies have also highlighted the inaccuracies of intraoperative inspection and palpation to detect nodal disease. The decision on whether to undertake lymphadenectomy should not be based on palpation of the nodal area, because less than 10% of patients with nodal metastases have grossly enlarged nodes. In addition, frozen section for histologic grade and depth of myometrial invasion in endometrial cancer correlates poorly with final pathology. In one study, on final pathology, 46% of patients with a confirmed uterine tumor on frozen section were upstaged and 38% have a higher histologic grade than reported by frozen section.

Therefore, there is a need to develop methods for the early detection of gynecological cancers, including a premalignant ovarian cancer condition which can be easily performed, for example in a gynecologist office, and a surgical technique that provides accurate pathologic information about the nodal status of patients with stage I endometrial cancer without over treating potentially low-risk patients and under-treating patients with metastatic disease. The present disclosure addresses these issues.

### SUMMARY OF THE INVENTION

The present invention overcomes the aforementioned drawbacks by providing a system and method to perform tubal lavage of a patient for ovarian cancer detection. The device includes a transvaginal probe configured to be inserted into a vagina of the patient, and a catheter and a needle both coupled to the transvaginal probe. The catheter is positioned relative to the transvaginal probe so that the catheter enters an endometrial cavity of the patient when the transvaginal probe is inserted into the vagina. The catheter is also configured to deliver fluid into the endometrial cavity, through fallopian tubes, and into a cul-de-sac of the patient. The needle is positioned relative to the transvaginal probe so that the needle enters the cul-de-sac when the transvaginal probe is inserted into the vagina and is configured to collect the fluid delivered by the catheter from the cul-de-sac.

In accordance with one aspect of the invention, a method for ovarian cancer screening of a patient includes inserting a catheter into an endometrial cavity of the patient, injecting a fluid into the endometrial cavity using the catheter, and at least partially occluding the endometrial cavity so that the fluid enters fallopian tubes of the patient, washes and collects cells as it flows through the fallopian tubes, and flows into a cul-de-sac of the patient. The method also includes inserting a collection needle into the cul-de-sac, collecting the fluid containing the cells from the cul-de-sac, and analyzing the cells for indications of ovarian cancer.

In accordance with another aspect of the invention, a device for ovarian cancer screening is disclosed. The device includes a transvaginal probe, a catheter coupled to an anterior surface of the transvaginal probe by at least one first coupling element, and a needle coupled an opposite, posterior surface of the transvaginal probe by at least one second coupling element. The catheter extends in a substantially curved manner past an end of the transvaginal probe, and the needle extends substantially straight past the end of the transvaginal probe.

In another aspect, the present invention provides a system and method for early detection of ovarian cancer. The system includes a catheter that can pass through the vagina and deliver fluid into the endometrial cavity and the fallopian tubes. The catheter is support by an attachment to a trans-vaginal ultrasound probe that holds and guides a sampling needle into the cul-de-sac to collect the fluid that has been passed through the catheter and into the fallopian tubes. By passing into the fallopian tubes, the collected fluid contains cells that can be studied for signs of ovarian cancer.

Another aspect of the present invention provides a system and method for sentinel lymph node detection in endometrial carcinoma. The system includes a catheter that can pass through the vagina and deliver a fluid dye into the endometrial cavity. Once the dye is instilled into the endometrial cavity, it will be picked up by the tumor more rapidly than normal tissue (i.e., due to increased blood supply and permeability of cancers) and follow the lymphatic channels leading to the sentinel node.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a partial cross-sectional view of a device constructed in accordance with a first embodiment of the present invention, inserted into a body of a patient;

FIG. 2 is a partial cross-sectional view of a catheter inserted into a body of a patient;

FIG. 3a is a partial cross-sectional view of the device of FIG. 1 including a directional distal catheter tip inserted into a body of a patient; and

FIG. 3b is another partial cross-sectional view of the device of FIG. 1 including a circumferential distal catheter tip inserted into a body of a patient.

FIG. 4 is a flow chart illustrating an exemplary method in accordance with one embodiment of the present invention.

FIG. 5 is a partial cross-sectional view of a device, according to another embodiment of the present invention, inserted into a body of a patient;

FIG. 6 is another partial cross-sectional view of the device of FIG. 5 injecting fluid into the body of the patient;

FIG. 7 is a flow chart setting forth steps of an exemplary method in accordance with one embodiment of the present invention; and

FIG. 8 is another partial cross-sectional view of the device of FIG. 5 inserted into a body of a patient.

### DETAILED DESCRIPTION OF THE INVENTION

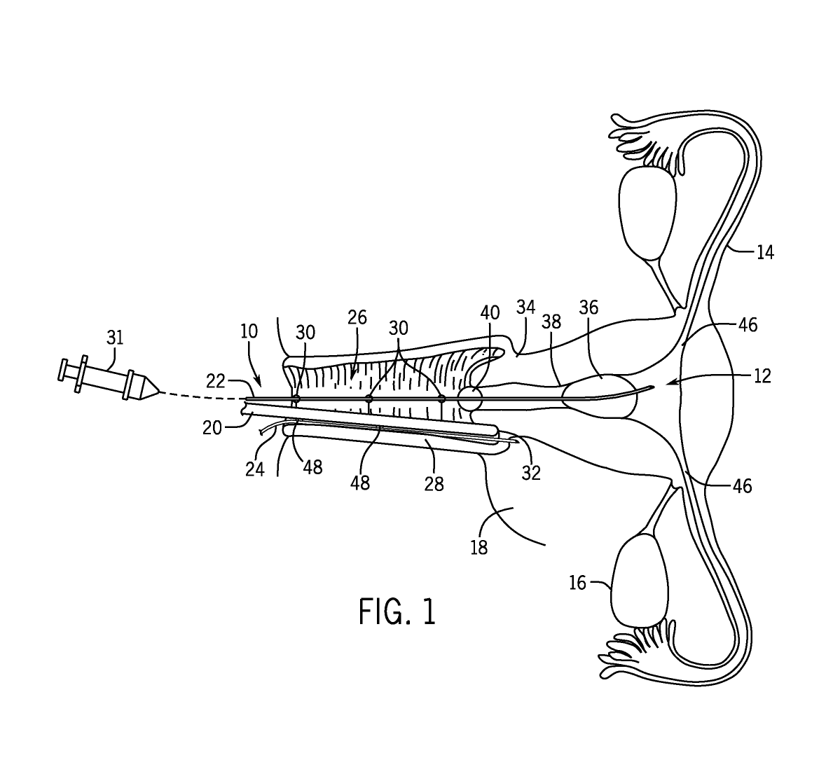

FIG. 1 illustrates a device 10 constructed in accordance with one embodiment of the present invention. The device 10 can be used to perform a tubal lavage by injecting a fluid in a patient's endometrial cavity 12 so that the fluid flows through the patient's fallopian tubes 14, over the patient's ovaries 16, and into the patient's recto-uterine pouch, or similar cul-de-sac 18. The fluid washes surfaces of the endometrial cavity 12 and the fallopian tubes 14, which loosens cells from the surfaces, so that the cul-de-sac 18 receives a solution of injected fluid and cells. The device 10 can collect the solution from the patient's cul-de-sac 18 so that the collected cells can be analyzed to determine if any are atypical, precancerous, or malignant cancerous cells and diagnose epithelial ovarian cancer (EOC).

As shown in FIG. 1, the device 10 can include a transvaginal probe 20, a catheter 22 coupled to the probe 20, and a collection needle 24 coupled to the probe 20. The transvaginal probe 20 can be inserted into the patient's vagina 26 in a conventional manner for a transvaginal ultrasound exam, for example abutting the patient's cervix 34. The catheter 22 can be coupled along an anterior surface of the transvaginal probe 20 so that the catheter 22 extends into the endometrial cavity 12 when the transvaginal probe 20 is inserted into the vagina 26. The collection needle 24 can be coupled along a posterior surface of the transvaginal probe 20 so that the collection needle 24 pierces the vaginal wall 28 and extends into the cul-de-sac 18 when the transvaginal probe 20 is inserted into the vagina 26. During operation of the device 10, the catheter 22 is used to the perform the tubal lavage, as described above, and the collection needle 24 is used to collect the mixture of injected fluid and collected cells from the tubal lavage.

As described above, the catheter 22 is coupled to the transvaginal probe 20, for example via coupling elements 30 as shown in FIG. 1. The catheter 22 can extend past an end 32 of the transvaginal probe 20 so that, when the transvaginal probe 20 is inserted into the vagina 26, the catheter 22 extends past the cervix 34 and into the endometrial cavity 12. As shown in FIG. 1, the coupling elements 30 can be arranged so that the catheter 22 curves substantially upward and outward away from the end 32 of the transvaginal probe 20 to facilitate insertion of the catheter 22 into the endometrial cavity 12 when the transvaginal probe 20 is inserted into the vagina 26.

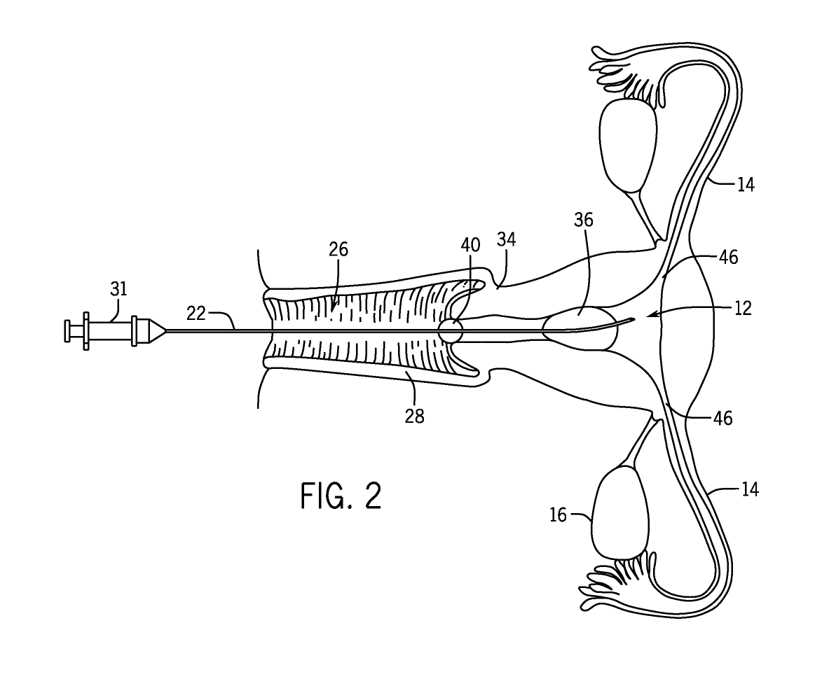

The catheter 22 can be of substantial length, for example about 40 centimeters, to allow external access to the catheter. This allows a syringe 31 to be coupled to an external end of the catheter 22 for injecting a fluid, such as saline, into the endometrial cavity 12. This part of the procedure is similar to what is performed during a conventional saline infusion sono-hystogram (SIS), a routinely performed diagnostic technique in gynecology where saline is injected through the cervix 34 and passes into the uterine cavity 12 transcervically to provide enhanced endometrial visualization during transvaginal ultrasound exam. Similar to SIS, fluid injection through use of the device 10 of the present invention can be easily and rapidly well performed in an out-patient setting, is well-tolerated by patients, and has minimal risk of complications. In addition, FIG. 2 illustrates a stand-alone catheter 22 inserted into the endometrial cavity 12 and connected to a syringe 31. The stand-alone catheter 22 can be used for injecting fluid into the endometrial cavity 12 to perform the tubal lavage described above, in accordance with the present invention.

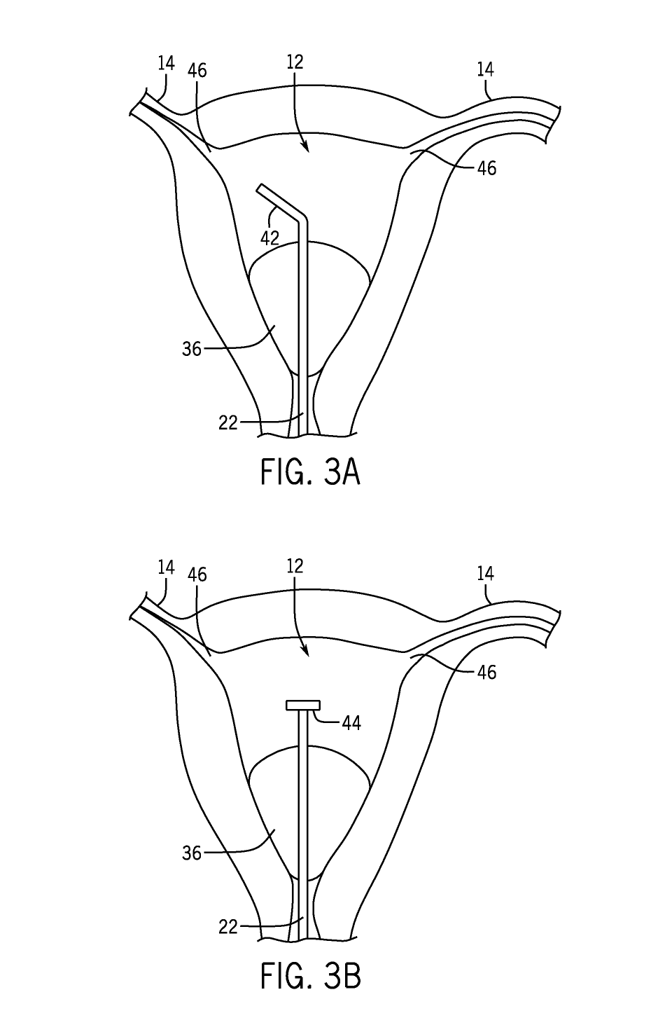

The catheter 22 can include an intra-endometrial cavity balloon 36 that at least partially occludes the endometrial cavity 12 at the distal endometrial lumen 38, as shown in FIG. 1. By at least partially occluding the distal endometrial lumen 38, the intra-endometrial balloon 36 forces the fluid introduced by the catheter 22 to flow through the fallopian tubes 14 and into the cul-de-sac 18. The intra-endometrial cavity balloon 36 can be constructed of a compliant material that can be inflated or filled to expand to a desired size or sizes. The desired size of the intra-endometrial cavity balloon 36 can be determined based on the degree desired for the injected fluid to contact the endometrial cavity lining or surfaces. The catheter 22 may also include an external balloon 40 positioned against the cervix 34 outside of the endometrial cavity 12, as shown in FIG. 1. In addition, the catheter 22 can include a directional distal tip 42 or a circumferential distal tip 44, as shown in FIGS. 3a and 3b, respectively. The directional distal tip 42 can be rotated so that fluid expelled by the catheter 22 can be directed in a specific direction, for example toward one of the fallopian tubes 14 (i.e., the proximal tubal opening or ostium 46 of one of the fallopian tubes 14). Using the circumferential distal tip 44, fluid expelled by the catheter 22 is directed outward in a multi-directional, circumferential manner, similar to a sprinkler head.

Referring back to FIG. 1, the collection needle 24 (e.g., a 20 gauge needle or similar) is coupled to the transvaginal probe 20 by coupling elements 48 and can extend past the end 32 of the transvaginal probe 20 so that, when the transvaginal probe 20 is inserted into the vagina, the collection needle 24 pierces and extends through the vaginal wall 28 and into the cul-de-sac 18. As shown in FIG. 1, the coupling elements 48 can be arranged so that the collection needle 24 remains substantially straight against the transvaginal probe 20 and extends straight outward from the end 32 of the transvaginal probe 20 to facilitate insertion of the collection needle 24 into the cul-de-sac 18 when the transvaginal probe 20 is inserted into the vagina 26. The collection needle 24 remaining substantially close against the length of transvaginal probe 20 can also prevent additional damage to the vaginal walls 28 during insertion of the transvaginal probe 20. The collection needle 24 is then used to collect the fluid, containing diagnostic cells from the fallopian tubes 14, that has been expelled into the cul-de-sac 18. The cells can be analyzed for abnormalities or indicators to screen for ovarian cancer. For example, the cells can be studied using modalities similar to a Pap smear, for increased PLA2 activity, or for molecular analysis of pre-malignant changes, such as BrCa1 mutations. In one specific example, the cells can be processed to prepare Haematoxilin and Eosin (H&E) stained slides, which can be evaluated for signs of atypia by a clinical pathologist.

In light of the description above, a method according to the present invention includes inserting the device 10 transvaginally (at process block 50), causing the transvaginal probe 20 to extend into the vagina 26, the catheter 22 to extend into the endometrial cavity 12, and the collection needle 24 to extend into the cul-de-sac 18. The method also includes injecting a fluid (e.g., 5-10 cc of saline) into the endometrial cavity 12 through the catheter 22 (at process block 52) and at least partially occluding the endometrial cavity 12 (at process block 54), for example using the intra-endometrial cavity balloon 36, so that the injected fluid enters and flows through the fallopian tubes 14 and the ovaries 16 and into the cul-de-sac 18. The injected fluid will collect cells as it washes the fallopian tubes 14 so that the fluid flowing into the cul-de-sac 18 includes the collected cells. The collection needle 24 is used to collect the fluid (at process block 56) and collected cells from the cul-de-sac 18, and the cells can then be analyzed for indications of ovarian cancer (at process block 58), as described above.

In accordance with the present invention, the purpose of the saline tubal lavage described above is to collect diagnostic cells from the fallopian tubes (and/or the endometrial cavity and the ovaries) because it is thought that, in ovarian cancer, cancerous cells originate in the fallopian tube. During the diagnosis of endometrial cancer, cancer cells have conclusively been retrieved from hysteroscopic fluid forced into the uterus and collected in the peritoneal cavity, illustrating the feasibility of the tubal lavage procedure of the present invention providing accessible cells from the fallopian tubes as a viable screening option for women at risk of ovarian cancer. Furthermore, ductal lavage is one of many minimally invasive breast cancer screening techniques and is used to collect cells from the milk ducts, where most breast cancer tumors are thought to originate.

The procedure described above can be carried out in an out-patient setting, for example in an gynecologist office. Further, this procedure does not impose the risks associated with the conventional surgical methods required for diagnosing ovarian cancer. Fallopian tube washing is already a common procedure in infertility evaluations. For example, fallopian tubes are washed with a radio-opaque, x-ray visible, or ultrasound imaging enhancing fluid media and then imaged to determine if a tubal occlusion is present, leading to a diagnosis of infertility.

Due to the present procedure being minimally invasive and presenting minimal risks, the procedure can be used to diagnose women with suspected ovarian cancer, or could potentially be used as a preventative testing procedure. Accordingly, the methods of the present invention offer a minimally invasive screening modality for the early detection of ovarian cancer (e.g., epithelial ovarian cancer) either as a premalignant condition, in situ, or metastatic disease. This can increase ovarian cancer detection at earlier stages and therefore greatly increase life expectancy of diagnosed patients.

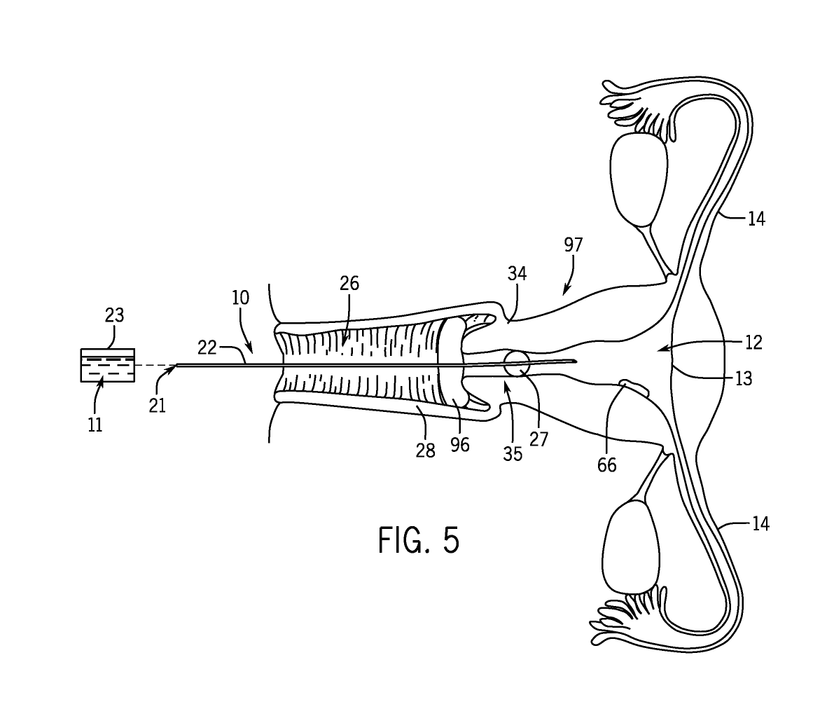

FIG. 5 illustrates a device 10 in accordance with another embodiment of the present invention. The device 10 can be used to deliver a task-specific fluid, such as a dye 11, into a patient's endometrial cavity 12, as shown in FIG. 2. The dye 11 contacts and is slowly absorbed by the endometrial lining 14. A tumor 66 present in the endometrial cavity 12 (i.e., an endometrial carcinoma) will absorb the fluid more quickly than the normal tissues of the endometrial lining 14 because of the increased blood supply and permeability of tumors. The first site of lymphatic drainage from the tumor 66 (i.e., through lymphatic channels 19) will be at the sentinel lymph node or nodes (not shown). In order to detect the sentinel node or nodes, a physician or surgeon can physically inspect the patient's lymph nodes for signs of staining by the dye.

If the carcinoma has metastasized, the cancer will have spread first to the sentinel node or nodes because these are the first nodes in a regional lymphatic basin that receive lymph flow from the tumor 66. Therefore, the sentinel lymph nodes can be biopsied first (i.e., prior to additional lymph node biopsies) and examined for the presence of cancer, as the histologic status of the sentinel lymph nodes can accurately predict the status of the regional lymphatic basin. If the cancer has spread to the sentinel node or nodes, additional therapies can be performed. If the sentinel lymph nodes are not identified as cancerous, there is a high likelihood that the cancer has not spread to any other areas of the body and additional lymphadenectomies may be deemed unnecessary. Therefore, by only removing one or two sentinel nodes, the complications of removing the typical 10-30 nodes can be avoided. For example, avoiding systematic or complete pelvic lymphadenectomies decreases morbidity due to increased operative times, leg lymphedema, infected or symptomatic pelvic lymphocysts, and/or chylous ascites associated with such procedures.

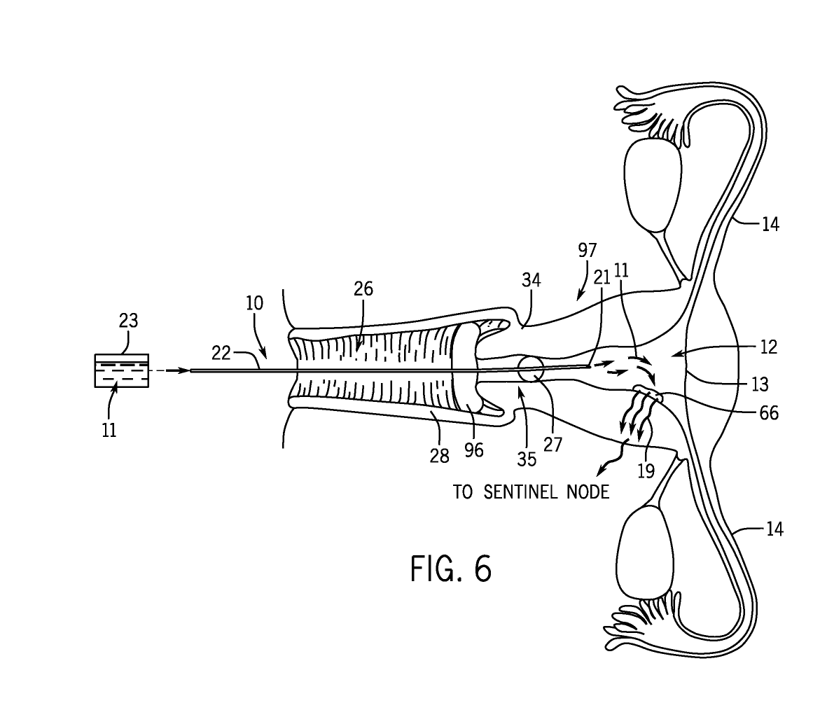

As shown in FIGS. 5 and 6, the device 10 can include a catheter 22 having an internal lumen 21 and configured to be inserted through a patient's vagina 26 and cervix 34 and positioned within the patient's endometrial cavity 12. The catheter 22 is designed to be coupled with a fluid reservoir 23 that holds the dye 11. The catheter 22 is coupled to the fluid reservoir 23 to inject the dye 11 transcervically into patient's endometrial cavity 12. One exemplary suitable dye in accordance with the present invention is Isosulfan blue (Lymphazurin™). Isosulfan blue is a synthetic visual lymphatic agent, with a low incidence of adverse reactions, that is typically injected into the periphery of a tumor site and localizes to the lymphatic system, causing sentinel nodes to stain blue for surgical identification.

An internal balloon 27 can be coupled along a length of the catheter 20 so that the internal balloon 27 is adjacent to the internal orifice 25 of the cervix 34 when the catheter 22 is positioned in the endometrial cavity 12. The internal balloon 27 acts to occlude the endometrial cavity 12 when the dye is injected. As shown in FIGS. 1 and 2, the internal balloon 27 may be relatively small in size to facilitate maximum exposure of the endometrial lining 13 to the injected dye. More particularly, the internal balloon 27 is specifically designed and sized to substantially avoid interfering with exposure of the endometrial lining 13 to the dye 11, particularly, along portions of the endometrial cavity 12 known to be associated with lymphatic channels 19. Thus, the catheter 22 and internal balloon 27 are specifically designed and sized to have a high probability of exposing the dye 11 to the tumor 66 and the sentinel lymph node or nodes.

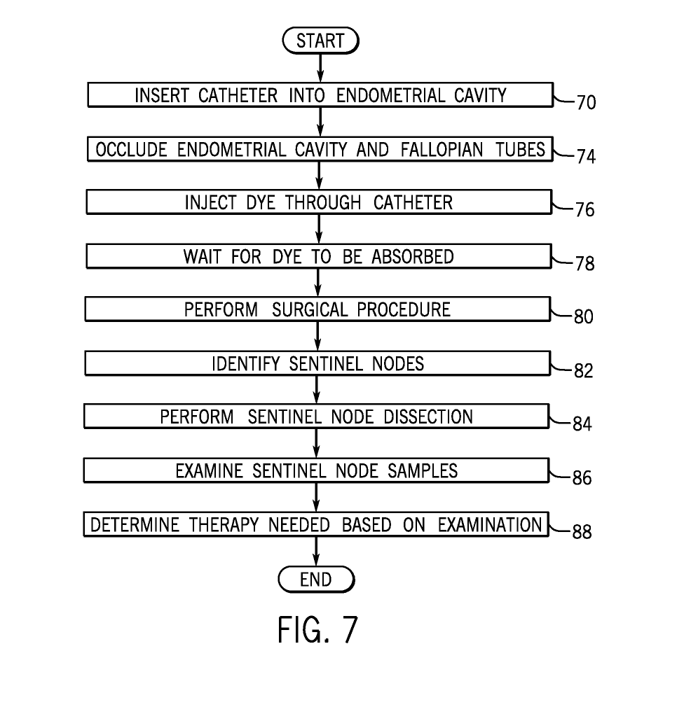

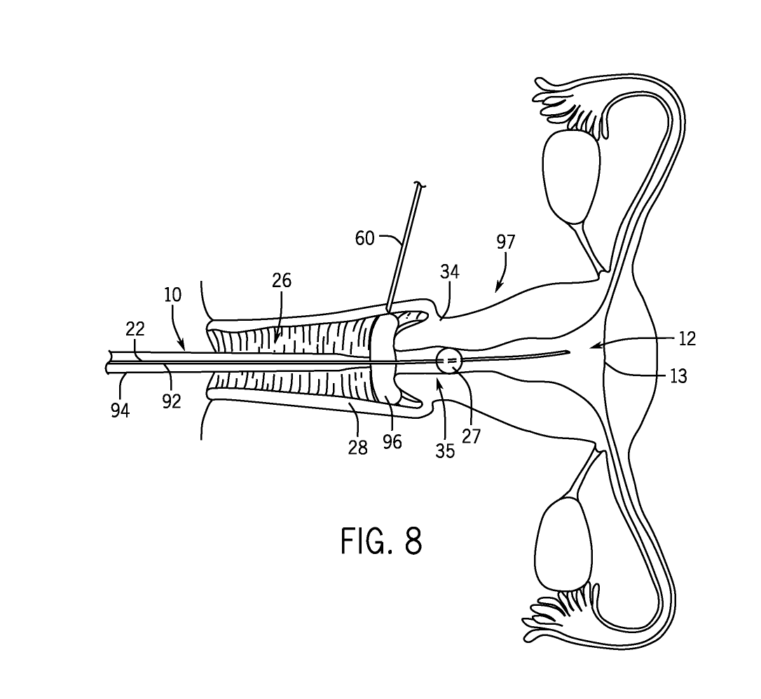

As described above, the device 10 can be used for sentinel lymph node mapping, for example in women with a preoperative diagnosis of stage I endometrial cancer. Currently, the use of sentinel lymph node mapping is only a well-established practice in the treatment of melanomas and breast cancer. Current sentinel node techniques and procedures for uterine cancers, including dye or tracer (i.e., radioactive isotope) injections into the cervix 34, into the endometrium during hysteroscopy, or to the subserosa during laparoscopy or laparotomy, are imprecise, unreliable and have a low sensitivity. For example, various combinations of these methods have resulted in detection rates that vary between 0% and 92%. Furthermore, the current methods are not convenient or do not reliably indicate the status of the cancer. The present invention provides a method, as shown in FIG. 7, that allows delivery of sentinel node dye directly to the tumor 66 by injecting dye transcervically into the endometrial cavity 12 and is easily, rapidly, and reliably well performed, well-tolerated by patients and devoid of complications.

As shown in FIG. 7, the method includes inserting the device 10 transvaginally until the catheter 20 has passed through the cervix 24 and into the endometrial cavity 12 (process block 70). The endometrial cavity 12 is occluded by the internal balloon 26 and the fallopian tubes 14 are also mechanically blocked or occluded, for example by ligation using an electrocautery device (process block 74). The method also includes injecting the fluid (e.g., about 5 milliliters of 1% isosulfan blue dye) transcervically into the endometrial cavity 12 via the catheter 22 (process block 76). About five to about ten minutes, or another suitable time length, is allowed for the fluid the be absorbed by the surrounding tissue and taken up by the lymphatic vessels, turning the sentinel lymph nodes blue (process block 78).

After the dye has been injected, during and/or after the five-to-ten minute time period, laparotomy or laparoscopic assisted surgery may be performed, such as for a total hysterectomy or bilateral salpingoophorectomy (process block 80). Following the surgical procedure, visual identification of dye coloring the sentinel lymph nodes is carried out (process block 82). The blue sentinel nodes are removed and a sample is taken from each blue node (process block 84). The samples are examined (process block 86), for example histopathologically with H&E (hematoxylin and eosin) or IHC (immunohistochemistry) or other staining or pathology practices to detect micrometastasis. Determinations for further therapy are then made based on the examination of the sentinel node samples (process block 88). For example, if no cancer is detected in the samples, further lymph node removal and testing may be deemed unnecessary. If micrometastasis is detected, further lymphadenectomies may be performed.

As described above, the device 10 allows for delivery of a dye transcervically into the endometrial cavity 12. Transcervical injection of a fluid, such as saline, is performed during a saline infusion sonohysterogram, or SIS. SIS is a common gynecological procedure where saline is injected into the endometrial cavity 12 to provide enhanced endometrial visualization during a transvaginal ultrasound exam. Unlike the SIS procedure, methods of the present invention call for injection of the dye into the endometrial cavity 12 and allowing the dye to be absorbed into the lymphatic system for surgical identification of sentinel nodes. The minimal complications of SIS and the fact that it is easily and rapidly performed and is well-tolerated by patients illustrates the feasibility of the method of the present invention as a well-tolerated, substantially low-risk procedure.

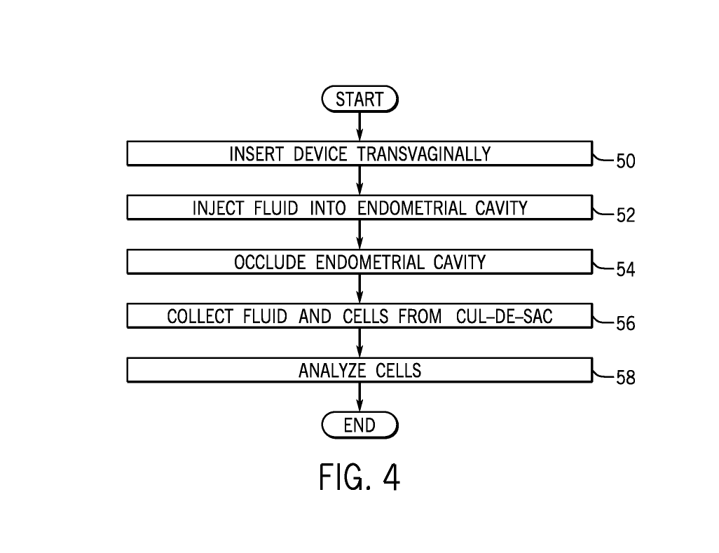

In some implementations of the present invention, the device 10 can also function as a uterine manipulator and/or a vaginal cuff incision guide, for example to assist in the surgical procedures described above with respect to process block 40. Uterine manipulation can be accomplished using a stiff, yet malleable rod or rods 72 inserted into the lumen of the catheter 22, as shown in FIG. 4, after dye injection has been completed. In addition or alternatively, the device 10 can include a stiff outer sheath 94 that can be advanced over a length of the catheter 22 to bestow enough rigidity to move the uterus 97 (e.g., to assist in a hysterectomy). The device 10 can also include an external balloon or guide 96. The guide 96 can be a large, durable sealing balloon coupled to the catheter 22 and positioned along the length of the catheter 22 to abut the cervix 34 when the catheter 22 is positioned within the endometrial cavity 12. When positioned, the guide can provide a visual or palpable landmark of an incision point to incise the vaginal wall 28 and perform a culdotomy for dissecting the cervix 34 and the uterus 97 from the vagina 26 (e.g., with a laparoscopic cutting needle 60) during a hysterectomy.

The present invention has been described in terms of one or more preferred embodiments, and it should be appreciated that many equivalents, alternatives, variations, and modifications, aside from those expressly stated, are possible and within the scope of the invention.

1. A device for screening for detection of gynecological cancers, the device comprising:
a transvaginal probe configured to be inserted into a vagina of the patient;
a catheter coupled to the transvaginal probe and positioned relative to the transvaginal probe so that the catheter enters an endometrial cavity of the patient when the transvaginal probe is inserted into the vagina, and
a needle coupled to the transvaginal probe and extending past an end of the transvaginal probe.

2. The device of claim 1 and further comprising an intra-endometrial balloon coupled to the catheter and configured to at least partially occlude the endometrial cavity so that the fluid delivered by the catheter flows from the endometrial cavity into the fallopian tubes.

3. The device of claim 1 wherein the catheter includes a directional distal tip capable of being rotated to direct the fluid in a single direction.

4. The device of claim 1 wherein the catheter includes a circumferential distal tip that directs the fluid circumferentially outward in multiple directions.

5. The device of claim 1 wherein the catheter is coupled to the transvaginal probe by at least one first coupling element, wherein the at least one first coupling element is arranged so that the catheter extends in a substantially curved manner past an end of the transvaginal probe

6. The device of claim 1 wherein the needle is coupled to the transvaginal probe by at least one second coupling element, wherein the at least one second coupling element is arranged so that the needle extends substantially straight past an end of the transvaginal probe.

7. The device of claim 1, wherein the catheter is configured to deliver fluid into the endometrial cavity, through fallopian tubes, and into a cul-de-sac of the patient. and the needle is positioned relative to the transvaginal probe so that the needle enters the cul-de-sac when the transvaginal probe is inserted into the vagina, and wherein the needle configured to collect the fluid delivered by the catheter from the cul-de-sac.

8. A method for screening a patient for ovarian cancer, the method comprising the steps of:
inserting a catheter into an endometrial cavity of the patient;
injecting a fluid into the endometrial cavity using the catheter;
at least partially occluding the endometrial cavity so that the fluid enters fallopian tubes of the patient, washes and collects cells as it flows through the fallopian tubes, and flows into a cul-de-sac of the patient;
inserting a collection needle into the cul-de-sac;
collecting the fluid containing the cells from the cul-de-sac; and
analyzing the cells for indications of ovarian cancer.

9. The method of claim 8 wherein the instilled fluid is saline.

10. The method of claim 8 wherein the step of analyzing the cells includes preparing stained slides of the cells and visually observing the stained slides.

11. The method of claim 8 further comprising rotating a directional tip of the catheter within the endometrial cavity so that the fluid is directed toward one of the fallopian tubes.

12. The method of claim 8 wherein the step of at least partially occluding the endometrial cavity includes inflating an intra-endometrial cavity balloon that is coupled to the catheter.

13. A system for sentinel lymph node detection of a patient with endometrial carcinoma, the device comprising:
a catheter configured to be inserted transvaginally into an endometrial cavity of the patient having an endometrial lining, the catheter having a lumen extending therethrough;
a fluid reservoir having a dye stored therein that is designed to be absorbed by the endometrial carcinoma and enter a sentinel lymph node of the patient, the fluid reservoir coupled to the lumen of the catheter to deliver the dye into the endometrial cavity; and
an internal balloon coupled to the catheter and positioned along a length of the catheter to occlude the endometrial cavity from a cervix of the patient when the catheter is positioned within the endometrial cavity, while substantially avoiding interfering with exposure of the endometrial lining to the dye along portions of the endometrial cavity commonly associated with lymphatic channels.

14. The system of claim 13 and further comprising an external balloon coupled to the catheter and configured to abut the cervix when the catheter is positioned within the endometrial cavity, the external balloon providing a visual and palpable guide during separation of the cervix from vaginal walls of the patient.

15. The system of claim 13 wherein the dye is an isosulfan blue dye.

16. The system of claim 13 and further comprising at least one rod configured to be inserted into the catheter after delivery of the dye, the at least one rod being movable to manipulate a uterus of the patient during a surgical procedure.

17. The system of claim 13 and further comprising sheath configured to be inserted over a length of the catheter, the sheath being movable to manipulate a uterus of the patient during a surgical procedure.

18. A method for sentinel lymph node detection in a patient with endometrial carcinoma, the method comprising the steps of:
inserting a catheter into an endometrial cavity of the patient;
occluding the endometrial cavity and fallopian tubes of the patient;
injecting a dye into the endometrial cavity using the catheter;
waiting a time period for the dye to be absorbed by the endometrial carcinoma and drain into at least one sentinel lymph node; and
identifying the at least one sentinel lymph node.

19. The method of claim 18 and further comprising the steps of:
dissecting the at least one sentinel lymph node;
examining the at least one sentinel lymph node for micrometastasis; and
determining additional therapies needed for the patient based on the examination.

20. The method of claim 19 wherein the step of examining the at least one sentinel lymph node for micrometastasis includes examining a sample of the at least one sentinel lymph node stained by one of hematoxylin and eosin and immunohistochemistry staining techniques.

21. The method of claim 19 wherein the step of determining additional therapies needed for the patient based on the examination includes determining that additional lymph node dissection is required if micrometastasis is identified in the at least one sentinel lymph node.

22. The method of claim 18 and further including performing one of a hysterectomy and a bilateral salpingoophorectomy after injecting the dye.

23. The method of claim 18 wherein the dye is an isosulfan blue dye.

24. The method of claim 18 wherein the step of occluding the fallopian tubes includes cauterizing the fallopian tubes.

25. The method of claim 18, wherein the time period is between about five minutes and about ten minutes.
